(19) United States
(12) Patent Application Publication
(10) Pub. No.: US 2025/0017718 A1
(43) Pub. Date: Jan. 16, 2025

(54) EXTRUSION DEVICE, METHOD FOR MANUFACTURING A HOLLOW STRUCTURE, AND USE OF AN EXTRUSION DEVICE

(71) Applicant: FRIEDRICH-ALEXANDER-UNIVERSITAET ERLANGEN-NUERNBERG, Erlangen (DE)

(72) Inventors: Christian LESKO, Erlangen (DE); Oliver FRIEDRICH, Erlangen (DE); Daniel GILBERT, Erlangen (DE)

(21) Appl. No.: 18/901,216

(22) Filed: Sep. 30, 2024

Related U.S. Application Data
(63) Continuation of application No. PCT/EP2023/052768, filed on Feb. 6, 2023.

(30) Foreign Application Priority Data
Mar. 28, 2022 (DE) 10 2022 203 033.8

(51) Int. Cl. A61F 2/06; B29C 48/09; B29C 48/21; B29C 48/25; B29C 48/335; B29L 23/00

(52) U.S. Cl. CPC A61F 2/06; B29C 48/09; B29C 48/21; B29C 48/2665; B29C 48/338; A61F 2240/001; B29L 2023/007

(57) ABSTRACT

An extrusion device for manufacturing a hollow structure by coaxial extrusion of a plurality of media. The extrusion device has a plurality of mountings, each for a nozzle for extruding one of the media. The mountings extend in an axial direction along a central axis and are arranged coaxially, for coaxial arrangement of the nozzles in one another. At least two of the mountings follow one another in a radial direction and are mounted against one another in the radial direction by use of a bearing such that the at least two mountings can be displaced relative to one another in the axial direction in order to adjust the relative axial positions of the respective nozzles.

### CROSS-REFERENCE TO RELATED APPLICATION

This application is a continuation, under 35 U.S.C. § 120, of copending International Patent Application PCT/EP2023/052768, filed Feb. 6, 2023, which designated the United States; this application also claims the priority, under 35 U.S.C. § 119, of German Patent Application DE 10 2022 203 033.8, filed Mar. 28, 2022; the prior applications are herewith incorporated by reference in their entirety.

### FIELD AND BACKGROUND OF THE INVENTION

The invention relates to an extrusion device, in particular for the biofabrication of a hollow structure. The invention furthermore relates to a method for manufacturing a hollow structure, and to the use of an extrusion device.

In biofabrication, one or more bioinks (for example cells dispersed in a hydrogel matrix) are used to manufacture a biostructure, for example an organ or a piece of tissue. Various methods for biofabrication are described for example in Zhang et al. 2015, entitled “In vitro study of directly bioprinted perfusable vasculature conduits”, Biomaterials Science, 3, 134-143, Liu et al. 2017, entitled “Synthesis of cell composite alginate microfibers by microfluidics with the application potential of small diameter vascular grafts”, Biofabrication, 9, 025030, Zhang et al. 2017, entitled “Microfluidic Bioprinting for Engineering Vascularized Tissues and Organoids”, J Vis Exp., Constantini et al. 2018, entitled “Co-axial wet-spinning in 3D bioprinting: state of the art and future perspective of microfluidic integration”, Biofabrication, 11, 012001, Liu et al 2019, entitled “Development of a Coaxial 3D Printing Platform for Biofabrication of Implantable Islet-Containing Constructs”, Adv Healthc Mater, e1801181, Jia et al. 2016, entitled “Direct 3D bioprinting of perfusable vascular constructs using a blend bioink”, Biomaterials, 106, 58-68, Cornock et al. 2014, entitled “Coaxial additive manufacture of biomaterial composite scaffolds for tissue engineering”, Biofabrication, 6, 025002 and Meng et al. 2016, entitled “Microfluidic generation of hollow Ca-alginate microfibers”, Lab Chip, 16, 2673-81. Further systems are described in U.S. Pat. No. 10,857,260 B2, U.S. patent publication No. 2016/0288414 A1, Chinese patent application CN 112 659 551 A, Chinese patent application CN 206 528 075 U and international patent document WO 2020/056517 A1.

One possible solution, in terms of equipment, for the biofabrication of a hollow structure is based on the use of medical cannulae or pipette tips as nozzles for coaxial extrusion, wherein the cannulae are aligned by hand and are joined together and sealed using adhesives. Although such solutions are relatively easy to use, they are generally manufactured only for one specific purpose, and furthermore typically also have poor surface quality, limited printing resolution and very large diameters. With the aforementioned nozzles, it is only possible to achieve minimum extrusion diameters of considerably greater than several 100 μm, typically of approximately 1 mm. Such solutions offer no possibilities for diameter minimization or flow optimization, and are therefore commonly not suitable for manufacturing small and/or complex hollow structures. In other words, there are accordingly resulting limitations with regard to the biostructures that can be manufactured, in particular with regard to the achievable structural, mechanical and rheological properties of the biostructure. Also, the process engineering equipment required for the described solutions is of prototype nature, and is at any rate not suitable for targeted, reproducible and automated manufacture, such as would be desirable for biofabrication—both for individual production and mass manufacture of biostructures. The described solutions also have little flexibility and can only be used to manufacture one particular biostructure. It is therefore also not possible to manufacture a complex biostructure, that is to say a biostructure having locally different shapes and/or properties, in particular branches.

### SUMMARY OF THE INVENTION

Against this background, it is an object of the invention to improve the manufacture of hollow structures, in particular in the context of a biofabrication process where the hollow structure manufactured is a biostructure consisting of a biomaterial. The manufacture should as far as possible be flexible, reproducible and automatable. For this purpose, it is sought to specify a correspondingly suitable extrusion device, a method for manufacturing a hollow structure, and the use of an extrusion device.

The object is achieved by an extrusion device having the features according to the independent extrusion device claim, by a method having the features according to the independent method claim, and by the use having the features according to the independent method use claim. The subclaims relate to advantageous embodiments, refinements and variants. The statements made in conjunction with the extrusion device also apply analogously to the method and to the use, and vice versa. Where steps of the method are specified below, advantageous embodiments of the extrusion device arise from the fact that said extrusion device is configured to carry out one or more of the steps, preferably automatedly.

The extrusion device serves for manufacturing a hollow structure (also referred to as hollow body) by coaxial extrusion of a plurality of media. Here, the media are each extruded and, in the process, in particular merged so as to jointly form, by combination and/or reaction with one another, the hollow structure. At least one of the media is preferably a biomaterial, such that the hollow structure is then accordingly a biostructure. It is furthermore preferable for at least one of the media to contain cells, the vitality of which remains ensured during the extrusion operation. This is however not imperative per se, that is to say the extrusion device described here is suitable both for the biofabrication of biostructures and for other fields of biotechnology, such as the manufacture of hollow fiber bioreactors or microchannel arrangements for a cell culture.

In one suitable embodiment, the extrusion device described here has three subassemblies, namely an extrusion assembly (also referred to as microfluidic assembly), a tool change assembly, and a drive unit (also referred to as linear unit).

In particular, the extrusion assembly forms a core element of the device and has multiple (that is to say at least two) brackets, each for one nozzle for extruding one of the media. Here, the nozzle is not part of the bracket but is in particular exchangeable, and is a consumable. The nozzles are however in particular part of the extrusion device as a whole. The brackets extend in an axial direction along a central axis and are arranged coaxially, in particular also concentrically, for coaxial arrangement of the nozzles one inside the other. At least two of the brackets, preferably all of the brackets, follow one another in a radial direction (that is to say perpendicularly to the axial direction) and are mounted on one another in the radial direction by means of a bearing, such that the at least two brackets are movable relative to one another in the axial direction in order to adjust the relative axial positions of the respective nozzles (that is to say the arrangement of the nozzles relative to one another). This is also referred to as “relative mobility”, “relative movement” or “axial relative movement” of the brackets/nozzles. In particular, one or more of the brackets has a corresponding bearing. The brackets are preferably movable relative to one another in steplessly variable or continuous fashion. The brackets are preferably movable relative to one another automatedly.

That bracket of the multiple brackets which is closest to the central axis is also referred to as the innermost bracket, and all of the other brackets are referred to as outer brackets in relation thereto. Correspondingly, that bracket of the multiple brackets which is furthest remote from the central axis is also referred to as the outermost bracket, and all of the other brackets are also referred to as inner brackets in relation thereto. Those brackets which are neither the innermost nor the outermost bracket are—if present—also referred to as middle brackets. Occasionally, the brackets are also numbered continuously from inside to outside, with the innermost bracket being the first bracket, the subsequent bracket being the second bracket, and so on. Accordingly, the same bracket may be designated differently depending on the context. The aforementioned designations also apply analogously to the nozzles, media and other components, which are provided in corresponding numbers.

Altogether, a nested structure is realized, in which the brackets with their respective nozzles are assembled along the axial direction so as to create a layered structure in a radial direction, through which the media can then be extruded concentrically during the course of the method. The brackets are each preferably cylindrical and/or rotationally symmetrical. The outer brackets are each suitably configured as hollow cylinders (that is to say so as to be generally U-shaped in cross section along the central axis) in order for one or more brackets to be correspondingly inserted therein.

The extrusion device has a front side and a rear side and generally extends in the axial direction from front to rear. The front side is correspondingly arranged at the front, where the nozzles are also arranged, the mouths of which are also situated at the front in order to manufacture the hollow structure there. In particular, the various media that are used for the manufacturing process are fed in at the rear.

The relative mobility of at least two of the brackets and thus also of at least two of the nozzles is implemented in particular by the mounting of the brackets on one another, that is to say the bearings fix the brackets relative to one another in the radial direction but allow a movement in the axial direction. In other words: at least two of the nozzles are movable independently of one another along the central axis. In particular, the brackets are movable during the course of the method, that is to say during the manufacture of the hollow structure, and are expediently also moved relative to one another during the manufacturing process in order to manufacture a hollow structure having locally different shapes and/or properties.

Each nozzle has, at the front, a mouth through which the associated medium ultimately emerges during the extrusion process. Viewed in the axial direction, there is then a spacing between the mouths of two nozzles, which spacing can be adjusted by moving the brackets. This yields various advantages: Firstly, owing to the relative mobility, it is possible to particularly precisely control the merging of the various media during the extrusion process and thus also the manufacture of the hollow structure, such that the manufacturing process is correspondingly flexible and it is also possible to manufacture complex hollow structures having locally different shapes and/or properties. The mechanical, geometrical, fluidic and rheological properties of the hollow structure are generally dependent on the interaction (for example reaction or mixing) of the media with one another, such that the aforementioned properties of the hollow structure can advantageously be set by controlling this interaction. This is readily apparent for example in the case of two media, one of which is a curing agent by which the other medium is cured upon contact. Owing to the relative mobility, the merging of the media and thus the interaction thereof are now precisely and reproducibly controlled, such that the aforementioned properties of the hollow structure are also correspondingly precisely and reproducibly set.

Additionally, owing to the relative mobility, axial production and/or assembly tolerances of the nozzles can also be particularly effectively compensated. Since the nozzles are consumables which are subject to corresponding production tolerances and are exchanged at regular intervals, it is accordingly possible to react to a change in length when exchanging a nozzle, which results in improved reproducibility in the manufacture of the hollow structure. Finally, the relative mobility in the axial direction in conjunction with the fixing in the radial direction also allows automation of the manufacturing process.

In order to compensate concentricity tolerances of the nozzles (that is to say tolerances of the nozzles in the radial direction), the brackets are expediently rotatable relative to one another about the central axis. Concentricity tolerances are then suitably compensated by rotating an outer bracket of the brackets, and thus also the nozzle thereof, relative to the next inner bracket such that the two concentricity tolerances are oriented radially in the same direction, and the effects thereof are thus minimized. After an assembly process has been completed, concentricity tolerances of two nozzles which follow one another in the radial direction are thus compensated by virtue of the two concentricity tolerances being oriented radially in the same direction. The concentricity tolerance is preferably measured at the mouth of each nozzle. Thus, for example, a concentricity tolerance at the mouth of an inner nozzle of 10 μm and a concentricity tolerance at the next outer nozzle of 12 μm gives rise, with an orientation having been adapted in this way, to a theoretical deviation of the concentricities of the mouths of only 2 μm.

In a particularly preferred embodiment, the multiple brackets are at least three brackets, that is to say the extrusion device has at least three brackets. As already described above, the brackets extend in the axial direction along the central axis and are arranged coaxially, for coaxial arrangement of the then at least three nozzles one inside the other. The brackets are furthermore preferably movable relative to one another in pairs in the axial direction in order to adjust the axial positions of the nozzles relative to one another. For this purpose, the three brackets follow one another, as described, in the radial direction and are mounted on one another in the radial direction, such that, viewed in the outward radial direction from the central axis, the three brackets/nozzles are an innermost, a middle and an outermost bracket/nozzle.

Here, without loss of generality, an embodiment as described above having three brackets and correspondingly also three nozzles for three media is taken as a basis, but the statements made apply generally to any desired number of brackets, nozzles and media (that is to say also only two nozzles for two media). An advantage of the invention is then also that the extrusion device is easily scalable by virtue of further brackets and nozzles simply being added as required, in order to manufacture hollow structures with any desired number of layers from a wide variety of media by extrusion.

In particular, each bracket is assigned exactly one nozzle, and in particular also exactly one medium, which is extruded by means of the nozzle. In one suitable embodiment, each nozzle consists of glass. Preferably, the nozzles are configured such that the hollow structure is manufactured with an internal diameter of at most 1 mm, preferably less than 500 μm, preferably less than 200 μm and particularly preferably in the range from 10 μm to 200 μm, particularly preferably 10 μm to 100 μm, or even only 10 μm to 50 μm. For this purpose, each nozzle correspondingly has, at least at the front, that is to say in the region of the mouth, an internal diameter of at most 1 mm, preferably less than 200 μm and particularly preferably in the range from 10 μm to 200 μm. In particular for this purpose, but also generally, an embodiment is suitable in which each nozzle is a drawn and/or reworked micronozzle consisting of a glass capillary. In other words, taking a glass capillary as a starting point, a nozzle is manufactured by virtue of the glass capillary being firstly heated and then drawn out in the axial direction, whereby its internal and external diameter are reduced toward the front. In this way, for example taking a glass capillary having an internal diameter of several 100 μm and an external diameter of 1 mm or more as a starting point, a nozzle having particularly small internal diameters, for example in the range of <200 μm, is then manufactured. Here, at least in the region close to and/or in the bracket, the original diameter of the glass capillary is maintained, such that the nozzle as a whole is accordingly tapered toward the front (that is to say at the front). The nozzles are in particular drawn outside the extrusion device, and independently thereof, using a separate drawing apparatus, such that the nozzle is firstly manufactured, and the fully drawn nozzle is then installed in the extrusion device. The nozzles are advantageously generally tapered as described, and thus of particularly streamlined form, which means in particular that as laminar as possible a flow of the particular medium, and a uniform pressure drop along the nozzles, are achieved during the extrusion process.

The question of what media are extruded, and using which of the nozzles, is basically arbitrary; these may be adapted as required. In one preferred embodiment, which is also taken as a starting point here without loss of generality, a first of the media is a support medium, in particular a cell suspension, for filling a lumen of the hollow structure; a second of the media is a wall material, in particular a biomaterial, for forming a wall of the hollow structure; and a third of the media is a curing agent (for example a crosslinker) for curing the second medium when merged with said second medium. It is preferable if the support medium is fed to and extruded by the first, innermost nozzle, the wall material is fed to and extruded by the second, middle nozzle, and the curing agent is fed to and extruded by the third, outermost nozzle. In the course of the manufacturing process, a wall of the hollow structure is then manufactured from the wall material by virtue of the wall material being brought into contact with the curing agent, such that the wall material cures. The curing agent is in this case brought into contact with the wall material from the outside. By contrast, the support medium is brought into contact with the wall material from the inside, and thus supports the wall material during the extrusion process.

A suitable wall material is hydrogel, for example alginate, and a suitable curing agent is calcium chloride (CaCl₂). A suitable support medium is water, but use is preferably made of a functional support medium, in particular a cell suspension, for example with endothelial cells for manufacturing blood vessels, or other cells (for example cell lines, stem cells, primary cells) for simultaneously functionalizing the wall from the inside during the manufacturing process, by virtue of cells being deposited on the inside of the wall in order to thus create a cell-coated hollow structure.

In a variant that departs from the embodiment described above, use is not made of a two-component system consisting of wall material and curing agent, and the wall is instead manufactured directly by extrusion of only a single medium. Accordingly, only two brackets, nozzles and media are then required. The use of a two-component system, however, has the advantage that it is thus possible to manufacture considerably smaller hollow structures than is possible using a single-component system. It is furthermore possible to use wall materials of very low viscosity, and to consequently avoid a high viscosity and thus a high shear load on living cells. Also suitable in principle is an embodiment in which the curing agent is brought into contact with the wall material from the inside, and then cures the wall material from the inside out. In this embodiment, the curing agent simultaneously serves as a support medium, such that it is also the case here that only two brackets, nozzles and media are required. Curing from the inside, for example in the described manner using a support medium that functions as a curing agent, may however also be combined with curing from the outside, for example by means of an external flow of a curing agent as described.

The extrusion device preferably has a combination of two or more of the four operating modes discussed below. A first operating mode is extrusion operation, in which the hollow structure is manufactured. A second operating mode is assembly operation, in which the individual brackets of the extrusion device, with installed nozzles, are inserted one inside the other. A third operating mode is a tool change mode, that is to say reception and fixing in the tool change assembly, and in particular the detachment and release thereof. A fourth operating mode is a fine adjustment mode, that is to say a fine adjustment of the nozzles in the axial direction in the assembled extrusion device in order to compensate for tolerances in the manufacturing process and in the fitting of the nozzles into the brackets.

A core concept of the invention is in particular a special mechanical design and configuration of the extrusion device such that it is made possible to manufacture particularly small, preferably even multi-layer hollow structures for the purposes of automated and preferably entirely additive biofabrication. This is achieved by the axial mobility of the nozzles relative to one another, such that the nozzles having minimal internal diameters, preferably of the order of magnitude of the diameter of a single cell (that is to say of ˜10 μm to 200 μm), can be reproducibly and precisely coaxially assembled. In the field of biofabrication, new production cycles are thus advantageously implemented, for example for the manufacture of perfusable branches.

Also, the extrusion device described here is suitable in particular for the coaxial microextrusion of particularly small, filamentous hollow structures consisting of cell-coated biomaterials. “Particularly small” is to be understood in particular to mean hollow structures having an internal diameter of less than 200 μm, preferably 10 μm to 100 μm, particularly preferably 10 μm to 50 μm. Manufacturing is performed for example with a processing speed in the range of 50 μm/s to 500 μm/s. A further advantage of the extrusion device according to the invention is that the coaxial microextrusion of biomaterials is not limited in terms of equipment, because the nozzles are selected according to requirements as a tool suited to a particular manufacturing step, is fixed in an associated bracket, and is axially finely adjusted by axial movement. Here, the coaxial orientation is advantageously not dependent on the technical skill of the user, but is intrinsically ensured by the design and manufacture of the extrusion device presented here. A reproducible manufacturing method is thus possible, which is fully automatable.

As already described, the extrusion device preferably has three subassemblies. Here, the extrusion assembly comprises the aforementioned brackets and allows the reception of the nozzles, the guidance and connection of the media to the nozzles, and in particular the precise, repeatably exact and adjustment-free installation of the nozzles, which are generally fragile. The mounting of the brackets on one another additionally allows the described relative mobility of the nozzles with respect to one another, in particular during operation, that is to say during the manufacturing process. Entirely new modes of operation and thus manufacturing possibilities are thus advantageously created, for example for the biofabrication of branched hollow structures, in particular hierarchical vascular systems, for example intended vessels and vessel networks. Furthermore, the brackets and the nozzles are advantageously also aligned exactly coaxially by means of the bearings, and therefore no centering has to be performed by the user. The bearing arrangement, and the design of the bearings, accordingly have particular importance in the present case.

The tool change assembly and the drive unit are then themselves initially optional, but advantageously realize a high level of automation and precision along with ease of use. For example, the tool change assembly makes it possible in particular for multiple extrusion assemblies to be changed automatedly, thus ensuring a fully automated and fully additive manufacturing process for biological microstructures. The brackets are preferably movable relative to one another automatedly by means of the drive unit, that is to say manual guidance of the nozzles is not necessary. In particular, the drive unit ensures the relative mobility of the nozzles in the axial direction during operation, the compensation of tolerances of the nozzles used in the axial direction, and highly precise, individual, automated control for the reception and fixing of an extrusion assembly during the tool change.

As already indicated, the axial mobility allows, as a new manufacturing step or cycle, the manufacture of one or more branches. In general, one of the brackets is an inner bracket and another of the brackets is an outer bracket. In one suitable embodiment, the inner bracket, preferably even the innermost bracket, is movable in the axial direction relative to the outer bracket to such an extent that the nozzle of the inner bracket protrudes relative to the nozzle of the outer bracket, specifically at the front, in order to pierce an existing hollow structure for the purposes of forming a branch by integral formation of a hollow structure on the existing hollow structure. The inner nozzle is thus used both to produce an opening in an existing hollow structure and as a placeholder for a lumen of a subsequently manufactured further hollow structure that is formed integrally on the existing hollow structure. Here, the axial mobility is utilized as follows: firstly, the inner nozzle is moved forward to such an extent that it protrudes out of the outer nozzle, that is to say the inner nozzle is deployed out of the outer nozzle. The existing hollow structure is then pierced (or perforated) by means of the inner nozzle by virtue of the inner nozzle being driven into a wall of the hollow structure. Then, if still necessary, the outer nozzle is pushed as far as against the wall of the existing hollow structure, and in the process the spacing between the mouths of the two nozzles is reduced again. Then, via the outer nozzle, the associated medium is extruded such that a connection to the existing hollow structure is made; in the process, the outer nozzle is if necessary pushed backward again, and the spacing of the mouths of the two nozzles is accordingly increased again. Here, the inner nozzle holds a connection to the lumen of the existing hollow structure open. From a particular point onward, which is marked for example by a specified spacing between the mouths of the two nozzles, no further relative movement takes place, and the inner nozzle is pulled out of the existing hollow structure and is guided concomitantly with the outer nozzle. The manufacture of the hollow structure is then continued in the normal way. The statements made apply analogously to the manufacture of the further hollow structure by means of multiple outer nozzles and brackets, for example to a two-component system for manufacturing the wall. The extrusion of the various media of the then multiple outer nozzles may then be performed in time-delayed fashion as required.

It is specifically the case in particular that the brackets, the mounting thereof on one another by means of suitable bearings and the relative mobility thereof form a core element of the present invention, and address a technically demanding range of problems, including: achieving as precise, reproducible and coaxial as possible an alignment of multiple nozzles for coaxial extrusion of a plurality of media for the purposes of manufacturing a hollow structure; achieving as simple, reproducible and destruction-free as possible an installation of the generally particularly fragile nozzles; and furthermore achieving a connection of the nozzles with correspondingly small internal diameters at the mouth to a macroscopic media connection, preferably fittings from the HPLC (high-performance liquid chromatography) field. This range of problems is addressed by the various embodiments and refinements of the extrusion device described here.

The brackets are preferably each configured as a rotationally symmetrical component. Each bracket has, in particular at the front, a receptacle (for example fit) for one of the nozzles, such that each nozzle is then installed at the front in an associated bracket. All of the receptacles lie in particular one behind the other on the central axis. Each receptacle is preferably configured as a transverse press fit, such that, if the bracket is heated, the thermal expansion thereof allows the nozzle to be inserted into the receptacle. Cooling then results in a high-strength, highly precise and sealed connection, which can advantageously also be released again by reheating. It is expedient if the nozzles, on the one hand, and the brackets, on the other hand, have coefficients of thermal expansion which differ to the greatest possible extent, thus making it possible for heating of the brackets to be performed slowly in a commercially available laboratory drying oven, eliminating the need for special induction devices, for example from the field of machine tools. A suitable material selection that exhibits significantly differing coefficients of thermal expansion is for example borosilicate glass for the nozzles and PEEK (polyether ether ketone) or rust-resistant steel for the brackets.

The innermost bracket forms a center of the extrusion assembly as a whole, and, by contrast to the other brackets, is of relatively solid form, preferably in the form of the solid cylinder and not merely a hollow cylinder. Multiple channels for guiding the various media are then preferably formed into said innermost bracket—irrespective of the preferred rotational symmetry. The channels are for example each formed as bores in the innermost bracket. The channels each ultimately lead to one of the nozzles in the various brackets. One of the channels leads firstly to the innermost nozzle; this channel suitably extends along the central axis and is therefore also referred to as a central channel. The other channels then extend eccentrically with respect to, that is to say with a radial spacing to, the central axis, and so as to be either parallel or inclined with respect to said central axis, depending on the dimensioning of the brackets and any structural space requirement for in particular standardized media connections. Each channel has, for example, a diameter of 0.5 mm to 1 mm. The media connections, for example HPLC fittings, are preferably formed at the rear on the innermost bracket. Alternatively, each of the brackets has a dedicated media connection that is connected via a channel in the corresponding bracket to the receptacle and to the nozzle that is fastened therein. The grouping of the media connections and channels in one of the brackets, in particular in the innermost bracket, is however preferred.

In an embodiment having three brackets/nozzles, it is expedient if one of the channels leads through the innermost bracket and opens, at the front thereof, into a head space which is formed in the axial direction between the innermost bracket and the subsequent middle bracket. In combination with the innermost nozzle, the head space is annular. The second medium, which emerges from the front of the innermost bracket via the channel, then enters the head space and is guided there from the outside along the innermost nozzle in order to ultimately emerge, in particular in annular fashion, through the corresponding middle nozzle. The same applies analogously to the next, outermost nozzle, with the difference that the associated third medium does not emerge from the front of the innermost bracket but expediently emerges from the side, that is to say in the radial direction, and is firstly guided into an annular channel in the middle bracket, from where the third medium is guided for example through one or more channels in the axial direction into a head space between the middle and outermost brackets. From there, the third medium is then, analogously to the second medium, guided in annular fashion between the middle and outermost nozzles, and ultimately discharged at the front.

As already described, at least two of the brackets, preferably all of the brackets, are mounted on one another by means of a bearing. Here, in each case two brackets which follow one another in the radial direction are mounted on one in particular by means of a single bearing. Accordingly, in the case of three brackets, two bearings are formed. Each bearing is suitably formed from a bearing inner surface and bearing outer surface that extends around the bearing inner surface. The bearing outer surface preferably lies in the radial direction against the bearing inner surface, such that the bearing is a plain bearing. The bearing outer surface is suitably a bearing bore. In the case of two brackets mounted on one another, the bearing bore is formed within the outer of the two brackets, and is in particular an internal wall of the bracket. The internal wall then corresponds to the bearing outer surface of the bearing. At the same time, the inner bracket is inserted into the bearing bore, giving rise to the aforementioned layered structure of the brackets. By contrast, the bearing inner surface is a part of an external wall of the inner of the two brackets. It is possible in principle for the bearing inner surface to correspond entirely or predominantly to the external wall, but an embodiment is preferred in which the bearing inner surface is formed only in certain portions, specifically only as one or more bearing regions in which the external wall has an enlarged diameter, that is to say the bearing inner surface protrudes radially beyond the rest of the external wall at one or more positions, forming bearing regions here (an equivalent to this is a reversed embodiment in which the bearing regions are formed as parts of the bearing outer surface, that is to say as annular and radially inwardly projecting regions of the internal wall of the outer bracket, though this is more difficult to manufacture). The bearing bore is a highly precisely machined bore, and the bearing inner surface has one or more correspondingly machined and preferably narrow load-bearing cylindrical regions, specifically the aforementioned bearing regions, as part of an external wall of the inner bracket in each case. Each bearing region is therefore in particular annular. Below, without loss of generality, bearings which each have multiple, in particular two, bearing regions will be taken as a basis. In a bearing that has multiple bearing regions, the bearing regions preferably have the same diameter. Each bearing serves in particular for allowing axial movement during extrusion operation and during fine adjustment operation and for stably holding the extrusion assembly together in the radial direction during tool change operation. Also of central importance, in particular, is the stabilization of the two brackets as they are placed one inside the other during the assembly operation.

Accordingly, in one suitable embodiment, the bearing serves for stabilizing and guiding the brackets and for coaxially aligning same as they are placed one inside the other during an assembly process (that is to say during the assembly operation). Here, the bearing has at least one bearing region which is configured and arranged such that, as the brackets are placed one inside the other, the bearing region always ensures guidance such that, in the radial direction, a clearance of an inner, in particular projecting nozzle of the nozzles does not in any region exceed a specified gap dimension in relation to an outer nozzle of the nozzles, so as to prevent damage to the nozzles during the placement one inside the other. Accordingly, the bearing region is positioned and configured so as to interact with the bearing bore of the bearing, during the placement one inside the other, at least if the specified gap dimension is undershot. In other words, the bearing is expediently configured such that damage to the generally very fragile nozzles is prevented to the greatest possible extent during the placement one inside the other. Owing to the anticipated small extrusion diameters, a minimal (and thus specified) gap dimension between the coaxially assembled nozzles during extrusion, tool change and fine adjustment operation is suitably always less than 200 μm, and often even only a few micrometers depending on the shape and diameters of the nozzles used. The bearings are thus sufficiently rigid and free from play to avoid, under all circumstances, asymmetrical positioning and thus irregular wall thicknesses during extrusion operation, or even contact between the nozzles, despite the aforementioned small gap dimensions. The fits between the bearing inner surfaces and bearing outer surfaces are thus advantageously configured as closely toleranced transition fits.

In one suitable embodiment, to keep friction low and improve the tribological properties, the bearing inner surface of at least one bearing is divided into two in particular narrow regions, which are situated as far apart as possible in order to nevertheless ensure the greatest possible rigidity. In other words, and more generally: the bearing has two bearing regions which are each in particular narrow and which are spaced from one another to the greatest possible extent in the axial direction. The bearing bore is in particular configured to be of such a length that the bearings exhibit sufficient rigidity. This means that at least part of the bearing inner surface (that is to say at least one of possibly several bearing regions) has entered the bearing bore when the mouth of the inner nozzle enters the shank region (rear portion of a nozzle) of the outer nozzle. The admissible radial play at the projecting tip (front portion of a nozzle) of the nozzle to the internal wall of the outer nozzle is based on the limited rigidity of the bearing arrangement and the tolerance of the fit, and is accordingly expediently smaller than the gap dimension between the inner and outer nozzles. At the latest when the mouth of the inner nozzle enters the relatively narrow drawn part of the outer nozzle, the entire bearing bears load by virtue of at least two and preferably all of the bearing regions having entered the bearing bore. By contrast, in the case of particularly small gap dimensions already in the shank region of the outer nozzle, it is suitably the case that at least two or all of the bearing regions already bear load when the inner nozzle enters the shank region.

The bearing preferably has at least two bearing regions, namely a front bearing region, which is arranged at the front in the axial direction, and a rear bearing region, which is arranged at the rear in the axial direction.

The front bearing region and the rear bearing region are arranged one behind the other, and spaced from one another, in the axial direction such that, at all times during the extrusion process (that is to say during extrusion operation), both bearing regions bear load, that is to say engage and make contact with the bearing outer surface. Sufficient guidance and rigidity of the bearing arrangement are thus ensured, and therefore the coaxial alignment of the assembled nozzles is ensured at all times.

In one particularly expedient embodiment, the front bearing region is interrupted, that is to say divided into two parts, in the axial direction, specifically into two subregions which are each in particular annular, by an annular groove. The insertion of the inner bracket into the outer bracket is thus facilitated, and jamming in the initial millimeters is avoided. Measured in the axial direction, the groove preferably has a length 0.5 times to 3 times the length of one of the two adjoining subregions of said front bearing region. The depth of the groove is not necessarily equal to the extent to which the bearing region projects relative to the rest of the external wall. The bearing arrangement is thus configured suitably according to the various operating modes. During extrusion operation, the brackets are moved relative to one another only to such an extent that, at all times, both the front and the rear bearing region are engaged, such that maximum stability is ensured during the extrusion. It is likewise the case during tool change and fine adjustment operation that both bearing regions are always engaged, in order to ensure maximum stability and protect the sensitive nozzles. By contrast, during assembly operation, the front bearing region firstly stabilizes the brackets as early as possible as they are placed one inside the other, with the rear bearing region then engaging only at a later point in time as the brackets are inserted further one inside the other.

In particular, during assembly operation, the outer of the two brackets has a maximum movement travel relative to the inner of the two brackets, which maximum movement travel preferably corresponds at least to a length of the nozzle of the outer bracket. To ensure maximum precision of guidance and maximum rigidity, the spacing of the two bearing regions of the bearing in the axial direction is furthermore selected to be as great as possible. The spacing of the two bearing regions in conjunction with a projecting length of the bearing bore of the outer of the two brackets beyond the rear bearing region define, in particular, the maximum movement travel between the two brackets during assembly operation with full guidance of the bearing (that is to say when all of the bearing regions are engaged). This then determines the maximum possible length of the drawn region of the outer nozzle, and in the case of small gap dimensions between the inner and the outer nozzle in the shank region, even the maximum length of the entire outer nozzle.

The required and utilized movement travel is in particular dependent on the operating mode. In particular, the maximum movement travel in the operating modes differs by several orders of magnitude, with the maximum movement travel during extrusion operation being smaller than that during fine adjustment operation and considerably smaller than that during assembly operation. During extrusion operation, the maximum movement travel is preferably a few micrometers to a few hundred micrometers. During fine adjustment operation, the movement travel amounts to up to a few millimeters. During assembly operation, the maximum movement travel amounts to at least the entire length of the outer nozzle including the shank, that is to say several millimeters, suitably in the range from 10 mm to 40 mm. The bearings are each configured, by way of their two load-bearing bearing regions, so as to avoid statical overdeterminacy. The movement travels described here describe only the movement of the brackets relative to one another. By contrast, the entire extrusion device as a whole may in principle be moved to any desired extent, such that hollow structures of any desired length can be manufactured.

In general, the bearings described here are expediently each configured as plain bearings. The smallest possible tolerances of the bearings both with respect to one another and with respect to the central axis and the receptacles for the nozzles are expediently ensured by way of a suitable manufacturing process for the bearings, for example high precision turning and/or cylindrical grinding, lapping and honing. The surface quality or roughness depth thus achieved is chosen so as to achieve optimum tribological properties. Aside from the plain bearings mentioned above, a design of the bearings as hydrostatic or aerostatic bearings is also suitable; the statements made here, in particular relating to arrangement and dimensioning, also apply correspondingly to these and other types of bearings.

In one advantageous embodiment, one of the bearing regions functions as a sealing region, which further reinforces the requirements with regard to surface quality, roughness depth, small shape and dimensional tolerances, and close fits. To improve the sealing action, at least one of the bearings suitably has a bearing region which is divided into two parts by an annular channel for one of the media, such that another of the media, which enters the annular channel through a sealing gap of the bearing, is merged with the medium in the annular channel in order to seal off the sealing gap. This is particularly advantageous if a bearing region that is divided into two parts by a narrow groove is provided in any case for the purposes of stabilization during assembly operation, as described above, such that the bearing region is then likewise used for sealing purposes. The sealing gap is formed between the bearing inner surface and bearing outer surface which, despite bearing against one another, typically do not do so particularly tightly. The annular channel mentioned here therefore preferably corresponds to the annular channel for the second medium in the case of three brackets/nozzles, as already described further above. The sealing is realized in particular by the formation of a reactive seal as the two media are merged. The bearing region that is divided into two parts is preferably the front bearing region already mentioned above. The two aforementioned media are preferably the aforementioned wall material and the curing agent, which react upon contact such that the wall material cures and thus forms a reactive seal that seals off the bearing. Such a seal is particularly expedient as internal diameters of the nozzles become smaller, because a higher pressure is thus required to extrude the media, and the risk of leakage through the bearing region increases.

In the exemplary embodiment having three brackets, the innermost bracket preferably has the front bearing region, divided into two parts, for sealing purposes and for mounting on the middle bracket. The third medium, preferably the curing agent, is guided laterally out of the channel of the innermost bracket into the annular channel, in order to ultimately be guided from there into the head space between the middle and outermost brackets. Correspondingly, the annular channel is delimited in the radial direction by the external wall of the innermost bracket and the internal wall of the middle bracket, and in the axial direction by two partial surfaces of the front bearing region. The front partial surface of the front bearing region forms, together with the internal wall of the middle bracket, the sealing gap which connects the annular channel to the head space between the inner and middle brackets. The second medium is guided into the head space. Then, in the event of leakage, merging of the first and the second medium is possible via the sealing gap. In the event of a possible leak of the bearing, the bearing is then sealed off at least in the region of the sealing gap at the front partial region of the bearing region by the reactive seal that is formed. Aside from the embodiment described here, other configurations are however also conceivable; for example, the two media may be interchanged. The embodiment proposed here, in which the curing agent is extruded via the outermost nozzle, however has the advantage that a leakage flow of the curing agent is less disadvantageous than a leakage flow of the wall material, and therefore the sealing of only the front bearing region by means of the reactive seal is sufficient, and no additional sealing of the rear bearing region is necessary.

The front bearing region, irrespective of whether or not it is in two parts, is preferably formed as close as possible to an end side of the particular bracket, that is to say is arranged directly flush with the end side of the bracket, or is arranged at least in the first 1 to 3 mm of the external wall. This has the advantage that, already at a particularly early point in time as one bracket is placed onto the other bracket, statically determinate guidance of the two brackets relative to one another is ensured, and jamming is prevented, by means of the bearing. This advantage is even more pronounced in the case of the aforementioned two-part bearing region, specifically irrespective of the delimitation of the annular channel and the described seal.

In a likewise advantageous embodiment, a separate sealing region is arranged in front of the two bearing regions in the axial direction. This is preferably implemented in order to reduce the head volume between the two brackets, that is to say in order to realize a smaller diameter of the liquid-guiding head region and thus of the dead volume in the extrusion assembly. In order to avoid statical overdeterminacy, the sealing region is expediently configured in terms of design and material to be considerably softer and more flexible than the bearing region. This is implemented for example by way of relatively narrow contact-making regions in the axial direction, or by introducing an additional dynamic seal consisting of relatively soft material. Correspondingly to the external geometry of the inner bracket in this sealing region, which, irrespective of the smaller diameter, may otherwise be implemented as described above, the outer bracket also has situated therein a bore which is arranged in front in the axial direction and which has a corresponding diameter for the outer sealing regions. The movement travel of the sealing region suitably corresponds at least to the required movement travel during extrusion operation and fine adjustment operation.

If the reduced diameter in the seal region of the bracket is not sufficient for a transverse press fit, for example because there is not sufficient material available for purely elastic reversible clamping, the receptacle for the nozzle is expediently extended into the region of the bearing behind the additional sealing region. The associated nozzle is however thus lengthened in the shank region. The length of the bearing arrangement (that is to say in particular the spacing between the front and rear bearing regions) however remains dependent on the length of the nozzles, whereby the assembly as a whole is lengthened.

As already indicated, one of the brackets, preferably the innermost bracket, has a channel for one of the media, in particular along the central axis. The channel expediently has a constriction having a diameter corresponding to an internal diameter of the nozzle that is connected to the channel. The medium is thus transferred with particularly little shear from the channel to the nozzle. The constriction is for example simply a constant reduced internal diameter as viewed in cross section along the central axis, such that a simple step is formed in the channel as a transition to the constriction. Such a step can be produced particularly easily using corresponding drills. In conjunction with the step, an embodiment is also advantageous in which a hose that is intended to guide the medium into the bracket is pushed into the channel as far as the constriction and abuts against the step and then has the same internal diameter as the constriction and the nozzle, such that the medium does not experience any change in diameter whatsoever on its path into the nozzle. In particular, the internal diameter then changes for the first time along the course of the nozzle.

In one suitable embodiment, in each case two brackets that are mounted on one another are produced from different materials. In other words, the brackets are produced alternately from at least two different materials, for example PEEK, on the one hand, and rust-resistant steel, on the other hand. This yields optimum tribological properties, and the relative mobility is improved. This design is advantageous in particular if the brackets are mounted on one another by means of plain bearings. Alternatively, all of the brackets are manufactured from the same material, which yields increased stability. This is advantageous in particular in combination with hydrostatic or aerostatic bearings.

The extrusion assembly suitably has one or more shape features for an automated “tool change”, that is to say an automatable change of extrusion assemblies in the extrusion device. One suitable shape feature is for example an encircling groove for allowing form-fitting, easily releasable accommodation in a magazine, for example in a rotary tool magazine with holding clamps.

The tool change assembly serves in particular as a holder device for the extrusion assembly during operation (in particular extrusion and fine adjustment operation), that is to say during the manufacture of a hollow structure and the preparatory fine adjustment. The tool change assembly clamps the brackets in particular together with the linear unit such that said brackets are movable precisely relative to one another in the axial direction as required, and are otherwise optimally fixed and centered relative to the linear unit. For this purpose, in a suitable embodiment, the extrusion device, in particular the tool change assembly, has a clamping device for each bracket for the purposes of holding and fixing the associated bracket. Each clamping device has a tension arm and a compression arm which peripherally engage around an associated bracket, such that an associated bracket is clamped in the axial direction between tension arm and compression arm. Accordingly, the phrase “peripherally engage around” is to be understood primarily to mean that the tension arm and the compression arm encircle the bracket and hold the bracket firmly between them.

The tension arm and the compression arm are in particular each tubular and are thus also referred to as tension tube and compression tube. In the case of the outer brackets, the compression arm is inserted into the tension arm, whereas in the case of the innermost bracket, the tension arm is conversely inserted into the compression arm. Each outer bracket then has a suitable peripheral contour that is clamped between the tension arm and the compression arm. This is however reversed in the case of the innermost bracket, where the tension arm pulls the bracket into the compression arm and thus firmly clamps the bracket. For this purpose, a groove or undercut is suitably also formed on the innermost bracket behind the peripheral contour thereof in order to allow the tension arm to engage therein. The tension arm and the compression arm of each clamping device are mounted on one another by means of a bearing, for example a plain bearing, and are movable relative to one another in the axial direction for the purposes of clamping the associated bracket. In particular, in each of the outer clamping devices, the compression arm is mounted in the tension arm, such that said tension arm can be moved exactly against the associated bracket by means of the drive unit in order to fix said bracket. The associated compression arm is moved together with the tension arm and then synchronously preloads the bracket without moving the corresponding bracket relative to the other brackets and thus undesirably extruding, or even drawing in, one of the media during the tool change (that is to say during assembly operation). The tension arms are each in particular connected to the drive unit, such that the relative mobility is ultimately realized by means of the tension arms. A movement of the compression arm relative to the tension arm for the purposes of preloading or releasing the associated bracket is realized for example by means of compressed air. The clamping is performed by means of the clamping device preferably at the front, whereas the bearings and compressed-air cylinders of said clamping device are suitably arranged at the rear.

In one suitable embodiment, the peripheral contour for clamping is formed by a cone of an associated bracket, and the clamping device has a corresponding internal cone. Here, in the case of the outer brackets, the internal cone is for example formed on the associated tension arm, and in the case of the innermost bracket, the internal cone is then conversely formed on the compression arm, such that the associated bracket is fixed and centered in the tool change assembly by axial tension. Furthermore, each cone, in particular of the outer brackets, expediently has a planar surface which extends in the radial direction and which is in particular correspondingly annular. The planar surface serves as a stop for the compression arm, such that it is then advantageously possible to preload the corresponding bracket and thus allow a play-free connection. Also suitable is a reversed embodiment in which, in the case of the outer brackets, the internal cone is formed on the associated compression arm and, in the case of the innermost bracket, the internal cone is conversely formed on the tension arm; in this case, each cone and the planar surface thereof are then analogously in a correspondingly reversed orientation. An angle of the cone and of the internal cone relative to the central axis is in this case selected such that it firstly does not have a self-locking action, but on the other hand the most effective possible centering is made possible. For example, an angle of 9°, and generally an angle in the range from 5° to 20°, is suitable.

The cone of the innermost bracket is preferably formed in the axial direction behind the aforementioned shape feature (for example groove) for the tool change. The cones of the outer brackets are expediently inclined oppositely to the cone of the innermost bracket, such that, owing to an internal pressure in each of the head spaces between the brackets, the innermost bracket and the outer brackets are forced in opposite directions and are thus clamped with greater intensity in the tool change assembly by way of the cones. Optimum fixing and centering are thus realized.

During tool change operation, the outer clamping devices are firstly unlocked by virtue of the associated compression arms and tension arms being moved apart, thus releasing the outer brackets. The extrusion assembly is then fixed only by the innermost clamping device. The extrusion device is then moved by the machine kinematics to a tool magazine or a tool changer, and the extrusion assembly is pushed, in particular at the above-described groove, into the holding clamp of the magazine. The final, innermost clamping device is thereupon unlocked, such that the extrusion assembly is fully released and is then held only by the holding clamps of the tool changer or magazine. The tool change assembly thereupon moves away from, and thus releases, the extrusion assembly. A correspondingly reversed procedure is followed in order to lock an extrusion assembly in the tool change assembly.

The clamping devices of the tool change assembly suitably each have, in the region of the extrusion assembly, a lateral cutout (also referred to as window) to allow lateral entry and exit of the extrusion assembly and media connections in the form of polymer hoses into and out of the tool change assembly and thus also allow a tool change. The cutouts suitably extend peripherally around one quarter to one third of a periphery of the associated clamping device.

The tension arms preferably swing freely and are suitably fixed and positioned in the axial direction by the aforementioned drive unit. The relative mobility of the brackets is in particular also implemented in this way. The drive unit is in particular configured such that axial tensile and compressive rigidity is ensured for the purposes of accommodating forces and achieving precise positioning, that is to say movement, whilst slight radial flexibility is at the same time provided in order to avoid statically overdeterminacy. This would give rise to radial forces on the bearings of the extrusion assembly and thus poor tribological behavior. The relative mobility of the brackets and clamping devices is implemented in particular by means of the drive unit, whereas a preload force of the clamping devices is implemented separately from the relative mobility, preferably pneumatically, for example by means of double-acting pneumatic cylinders. In other embodiments, a mechanical preload is also implemented, for example by means of suitable mechanical elements, for example threads or locking projections actuated by electric motors (stepper or servo motors).

The drive unit is suitably connected to the tension arms at least of the outer clamping devices of the tool change assembly, for example by means of screwed flanges. The drive unit is in particular as compact a connection as possible of two linear axes for implementing the relative mobility of the brackets. Since the extrusion device proposed here is, in one suitable embodiment, part of an additive production machine (for example of a bioprinter) and must regularly be moved as a whole by the machine kinematics of said production machine, as compact an embodiment as possible is advantageous. Furthermore, a force imparted by each of the two linear axes must be transmitted as exactly as possible in the axial direction into the extrusion assembly. In particular in the embodiment assumed here having three brackets/nozzles for a support medium at the inside, a wall medium in the middle and a curing agent at the outside, a relative movement of the innermost bracket with respect to the middle and outermost brackets is performed regularly during operation, whereas a relative movement of the outermost bracket with respect to the middle bracket is required relatively seldom by comparison. The same also applies to the described manufacturing cycle for the manufacture of branches in hollow structures. Therefore, in one suitable refinement, the drive unit is designed as a linear axis having a ball screw drive having a threaded spindle, which in particular has a shallow gradient, and having a fixed nut and a driven nut. Rotational fixing and linear guidance of the nuts is performed for example by means of a profiled rail guide. Drive is imparted for example by means of stepper motors and belt drives. Here, the fixed nut is connected by means of a suitable connection to the middle bracket, whereas the driven nut is connected, likewise by means of a suitable connection, to the outer bracket. To now move the innermost bracket relative to the other brackets, only the threaded spindle is driven. The relative positions of the other brackets with respect to one another are maintained in the process. By contrast, to move only the outermost bracket relative to the other brackets, the driven nut is moved, with the relative positions of the other brackets with respect to one another again being maintained. Drive is alternatively imparted by motors each having a hollow shaft or by means of rotary direct drives (for example torque motors) that are integrated into the drive unit. A construction having linear motors is also suitable in principle.

The extrusion device as a whole is advantageously configured such that, by adding further corresponding brackets to the extrusion assembly, further nozzles can be added, coaxially positioned, moved and operated. Correspondingly, further clamping devices are also added to the tool change assembly, and the drive unit is upgraded, for example with additional driven nuts. By means of such an expansion, it is then for example made possible to manufacture multi-layer microstructures for replicating relatively large blood vessels (>100 μm) with relatively complex morphology.

Suitable pumps (for example syringe pumps), valves (for example rotary valves) and connecting elements, for example from the field of chromatography, such as fittings and hoses, ensure in particular the supply of advantageously precisely metered, pulsation-free volume flows to the extrusion device. A pump controller suitable for this purpose is in particular connected or coupled to a controller of the drive unit in order, in the event of a relative movement of the brackets in the axial direction, to compensate the resulting change in volume of each head space, in particular in real time.

Other features which are considered as characteristic for the invention are set forth in the appended claims.

Although the invention is illustrated and described herein as embodied in an extrusion device, a method for manufacturing a hollow structure, and a use of an extrusion device, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a diagrammatic, side view of an extrusion device according to the invention;

FIG. 2 is another side view of the extrusion device from FIG. 1;

FIG. 3 is a perspective view of the extrusion device from FIG. 1;

FIG. 4A is a sectional view of an extrusion assembly of the extrusion device from FIG. 1;

FIG. 4B is a detail view relating to FIG. 4A;

FIG. 5A is a sectional view of a tool change assembly of the extrusion device from FIG. 1 in a locked state;

FIG. 5B is a sectional view of the tool change assembly from FIG. 5A in a partially locked state;

FIG. 5C is a sectional view of the tool change assembly from FIG. 5A in an unlocked state;

FIG. 6 is an illustration of the manufacture of a branch by means of the extrusion device from FIG. 1; and

FIG. 7 is an illustration of a hollow structure manufactured by means of the extrusion device from FIG. 1.

### DETAILED DESCRIPTION OF THE INVENTION

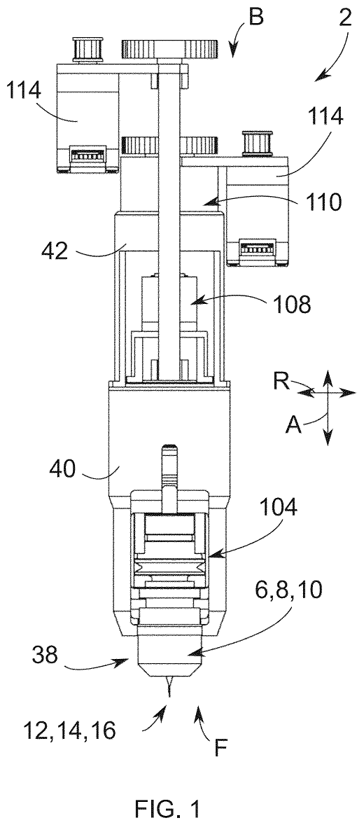

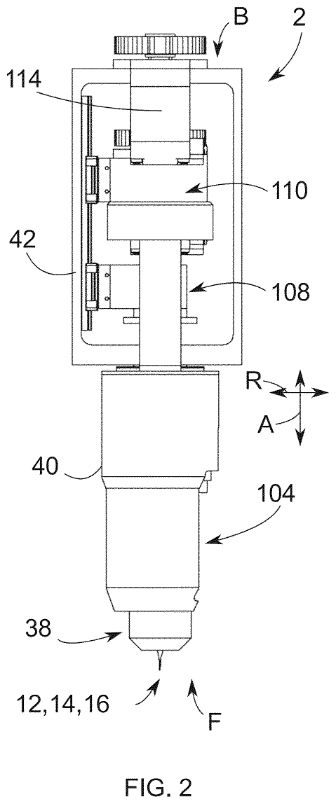

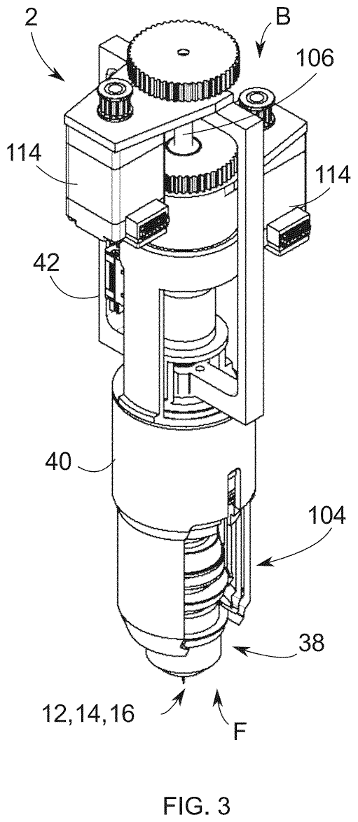

Referring now to the figures of the drawings in detail and first, particularly to FIGS. 1-3 thereof, there is shown, in various views, an exemplary embodiment of an extrusion device 2 that is used to manufacture a hollow structure 4 by coaxial extrusion of a plurality of media. Here, the media are each extruded and, in the process, merged so as to jointly form the hollow structure 4. An exemplary hollow structure 4 is shown in FIG. 7. In the exemplary embodiment shown, one of the media is a biomaterial, and at least one of the media contains cells, such that the hollow structure 4 is then a biostructure containing living cells. The extrusion device 2 described here is however suitable both for biofabrication and for other biotechnological fields, for example the manufacture of hollow fiber bioreactors or microchannel arrangements for a cell culture.

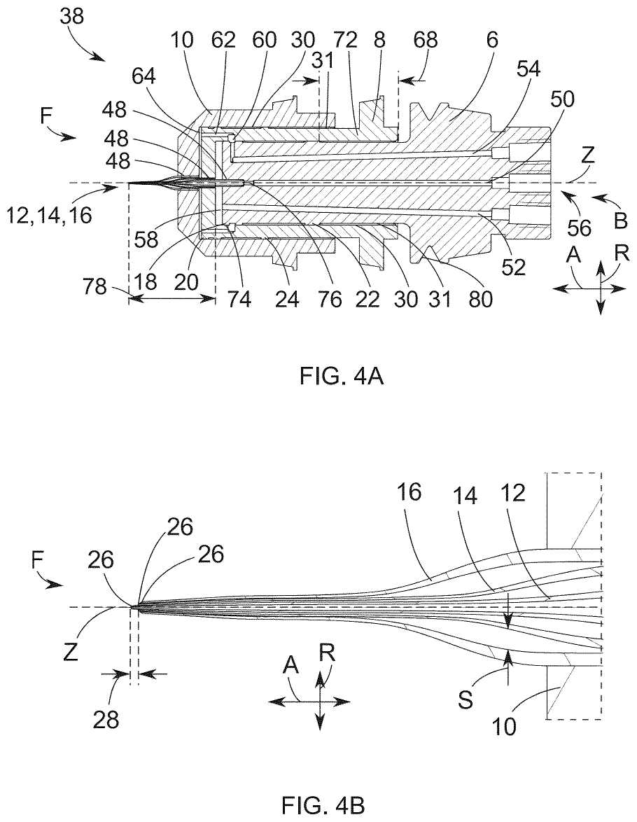

An extrusion assembly 38, for example as shown in FIG. 4A, is a subassembly of the extrusion device 2 and has multiple, in this case three, brackets 6, 8, 10, each for one nozzle 12, 14, 16 for extruding one of the media. This is shown in detail in FIG. 4A in a sectional view. Here, each nozzle 12, 14, 16 is exchangeable and is a consumable. FIG. 4B shows a detail view of the nozzles 12, 14, 16, which are arranged at the front F of the brackets 6, 8, 10. The brackets 6, 8, 10 extend in an axial direction A along a central axis Z and are arranged coaxially and concentrically, for coaxial arrangement of the nozzles 12, 14, 16 one inside the other. All of the brackets 6, 8, 10 follow one another in a radial direction R perpendicular to the axial direction A and are mounted on one another in the radial direction R by means of respective bearings, such that the brackets are movable relative to one another in the axial direction A in order to adjust the relative axial positions of the respective nozzles 12, 14, 16. This is also referred to as “relative mobility” or “relative movement”.

That bracket of the multiple brackets 6, 8, 10 which is closest to the central axis Z is also referred to as the innermost bracket 6, and all of the other brackets 8, 10 are referred to as outer brackets 8, 10 in relation thereto. Correspondingly, that bracket of the multiple brackets 6, 8, 10 which is furthest remote from the central axis Z is also referred to as the outermost bracket 10, and all of the other brackets 6, 8 are also referred to as inner brackets 6, 8 in relation thereto. Those brackets 8 which are neither the innermost nor the outermost bracket 6, 10 are—if present—also referred to as middle brackets 8. Occasionally, the brackets 6, 8, 10 are also numbered continuously from inside to outside, with the innermost bracket 6 being the first bracket 6, the subsequent bracket 8 being the second bracket 8, and so on. Accordingly, the same bracket 6, 8, 10 may be designated differently depending on the context. The aforementioned designations also apply analogously to the nozzles 12, 14, 16, media and other components, which are provided in corresponding numbers.

FIG. 4A shows the brackets 6, 8, 10 in a cross-sectional view along the central axis Z. As can be seen from FIGS. 4A and 4B, a nested structure is realized, in which the brackets 6, 8, 10 with their respective nozzles 12, 14, 16 are assembled along the axial direction A so as to create a layered structure in a radial direction R, through which the media can then be extruded concentrically during the course of the method. Here, the brackets 6, 8, 10 are each cylindrical and rotationally symmetrical. The outer brackets 8, 10 are each designed as hollow cylinders in order for one or more brackets 6, 8 to be correspondingly inserted therein.

The relative mobility of the brackets 6, 8, 10 and thus also of the nozzles 12, 14, 16 is implemented here by means of two bearings. Each of the bearings has a front bearing region 18, 20 and a rear bearing region 22, 24. In each case one front bearing region 18, 20 and one rear bearing region 22, 24 form a bearing inner surface, which lies against a bearing outer surface, which in this case is an internal wall 30 of an associated outer bracket 8, 10. Conversely, the bearing inner surfaces are a part of an external wall 31 of the associated inner bracket 6, 8. The bearings fix the brackets 6, 8, 10 relative to one another in the radial direction R but allow a movement in the axial direction A. This is possible in particular during the method, that is to say during the manufacture of the hollow structure 4, and is correspondingly utilized to manufacture a hollow structure 4 having locally different shapes and/or properties.

Each nozzle 12, 14, 16 has, at the front F, a mouth 26 through which the associated medium ultimately emerges during the extrusion process. For the sake of simplicity, the various mouths 26 are denoted here by the same reference sign. Viewed in the axial direction A, there is then a spacing 28 between the mouths 26 of two nozzles 12, 14, 16, which spacing can be adjusted by moving the brackets 6, 8, 10.

Although an embodiment having three brackets 6, 8, 10 and correspondingly also three nozzles 12, 14, 16 for three media is shown here, the statements made apply generally to any desired number of brackets 6, 8, 10, nozzles 12, 14, 16 and media. In fact, the extrusion device 2 is easily scalable simply by adding further brackets 6, 8, 10 and nozzles 12, 14, 16 as required.

In the embodiment shown, each nozzle 12, 14, 16 consists of glass and is designed such that the hollow structure 4 is manufactured with an internal diameter of 10 μm to 200 μm. For this purpose, each nozzle correspondingly has, at least at the front F, that is to say in the region of the mouth 26, a corresponding internal diameter. Each of the nozzles 12, 14, 16 shown here is a drawn and reworked micronozzle consisting of a glass capillary. Altogether, therefore, the nozzle 12, 14, 16 is tapered toward the front, as can be seen particularly clearly in FIG. 4B.

The question of what media are extruded, and using which of the nozzles, is basically arbitrary; these may be adapted as required. In the exemplary embodiment shown here, a first of the media is a support medium, in particular a cell suspension, for filling a lumen 32 of the hollow structure 4 and lining the internal wall thereof with cells; a second of the media is a wall material for forming a wall 34 of the hollow structure 4; and a third of the media is a curing agent for curing the second medium when merged with the second medium. The support medium is extruded by the first, innermost nozzle 12; the wall material is extruded by the second, middle nozzle 14, and the curing agent is extruded by the third, outermost nozzle 16. The wall 34 is then manufactured from the wall material by virtue of the wall material being brought into contact with the curing agent, such that the wall material cures. The curing agent is in this case brought into contact with the wall material from the outside. By contrast, the support medium is brought into contact with the wall material from the inside, and thus supports the wall material during the extrusion process. By way of example, the wall material is in this case a hydrogel, for example alginate; the curing agent is calcium chloride (CaCl₂); the support medium is a cell suspension, for example with endothelial cells in order to simultaneously functionalize the wall 34 from the inside during the manufacturing process, as shown in FIG. 7, by virtue of cells 36 being deposited on the inside of the wall in order to thus create a cell-coated hollow structure 4.

In a variant that is not shown, use is not made of a two-component system consisting of wall material and curing agent, and the wall 34 is instead manufactured directly by extrusion of only a single medium. Alternatively, the curing agent is brought into contact with the wall material from the inside, and then cures the wall material from the inside out. In this embodiment, the curing agent simultaneously serves as support medium.

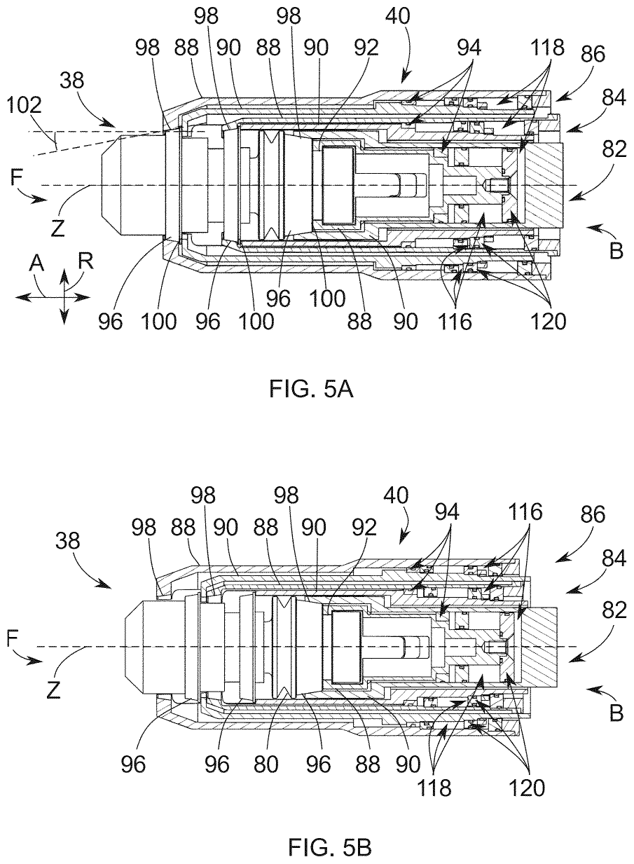

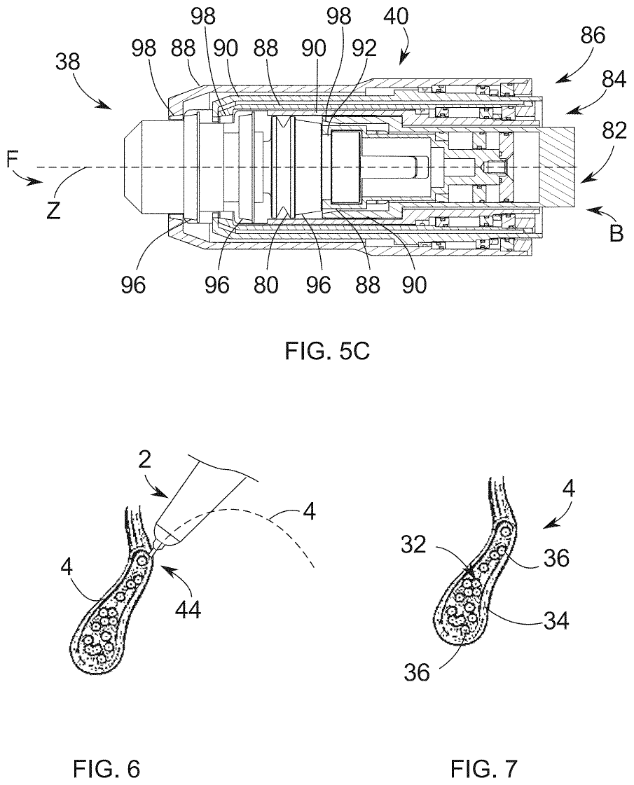

The extrusion device 2 described here has three subassemblies, namely an extrusion assembly 38, a tool change assembly 40 and a drive unit 42. Here, the extrusion assembly 38 contains the brackets 6, 8, 10 and allows the reception of the nozzles 12, 14, 16, the guidance and connection of the media to the nozzles 12, 14, 16, and the precise, repeatably exact and adjustment-free installation of the nozzles. The brackets 6, 8, 10 and the nozzles 12, 14, 16 are aligned exactly coaxially by means of the bearings having the bearing regions 18, 20, 22, 24. An exemplary extrusion assembly 38 has already been described in conjunction with FIG. 4A. An exemplary embodiment of the tool change assembly 40 is shown in FIGS. 5A, 5B and 5C in various states and likewise in a sectional view along the central axis Z (to provide a clearer illustration, the extrusion assembly 38 is not shown in section). An exemplary embodiment of the drive unit 42 can be seen in FIGS. 1, 2 and 3.

The tool change assembly 40 and the drive unit 42 are themselves optional for the basic function of the continuous extrusion but ensure high precision and reproducibility and are thus advantageous for fine adjustment operation, tool change operation and enhanced functions of the extrusion such as dynamic changes to the flow conditions, or the manufacture of branches. For example, the tool change assembly 40 makes it possible for multiple extrusion assemblies 38 to be changed automatedly, thus ensuring a fully automated and fully additive manufacturing process. The brackets 6, 8, 10 are preferably movable relative to one another automatedly by means of the drive unit 42, that is to say manual adjustment or fixing of the nozzles 12, 14, 16 during operation is not necessary. The drive unit 42 ensures the automated relative mobility of the nozzles 12, 14, 16 in the axial direction A during operation, the compensation of tolerances of the nozzles 12, 14, 16 used in the axial direction A, and highly precise, individual, automated control for the reception and fixing of an extrusion assembly 38 during the tool change.

The axial mobility allows, as a new manufacturing step or cycle, the manufacture of one or more branches 44, as illustrated by way of example in FIG. 6. When the extrusion device is used as described above, for this purpose, the innermost (also referred to as inner) bracket 6 is moved in the axial direction A relative to the middle bracket 8 (which, relative to the innermost bracket 6, is also an outer bracket 8) to such an extent that the innermost nozzle 12 protrudes relative to the middle nozzle 14, specifically at the front F, in order to pierce an existing hollow structure 4 for the purposes of forming the branch 44 by integral formation of a further hollow structure 4 (indicated by a dashed line in FIG. 6) on the existing hollow structure 4. The innermost nozzle 12 is thus used both to produce an opening in the wall 34 of an existing hollow structure 4 and as a placeholder for the lumen 32 of the subsequently manufactured further hollow structure 4 that is formed integrally on the existing hollow structure 4. For this purpose—if necessary—the middle nozzle 14 is pushed as far as against the wall 34 of the existing hollow structure 4, and in the process the spacing 28 between the mouths 26 of the two nozzles 12, 14 is reduced again. Then, via the middle nozzle 12, the wall material is extruded such that a connection to the existing hollow structure 4 is made. Here, the middle nozzle 14 is if necessary, pushed backward again, and the spacing 28 is accordingly increased again. Here, the innermost nozzle 12 holds a connection to the lumen 32 of the existing hollow structure 4 open. From a particular point onward, which is marked for example by a specified spacing 28, no further relative movement takes place, and the innermost nozzle 12 is pulled out of the existing hollow structure 4 and is guided concomitantly with the middle nozzle 14. Throughout this process, the outermost nozzle 16 for the curing agent is guided concomitantly with the middle nozzle 14, such that these two nozzles 14, 16 are not moved relative to one another. Then, proceeding from the start of the integrally formed branch, the extrusion device 2 as a whole is guided onward, whilst performing continuous extrusion, in a coordinated movement of the machine kinematics (bioprinter), whereby the lateral branch, as an independent hollow structure 4, can be extruded further as desired.

In the present case, the brackets 6, 8, 10 are each configured as a rotationally symmetrical component. Each bracket 6, 8, 10 has at the front F, a receptacle 48 for one of the nozzles 12, 14, 16, such that the nozzles are then each installed at the front in an associated bracket 6, 8, 10, as can be seen for example in FIGS. 4A-4C. All of the receptacles 48 are situated one behind the other on the central axis Z. The receptacles are in this case each designed as a transverse press fit.

The innermost bracket 6 forms the center of the extrusion assembly 38 as a whole, and, by contrast to the other brackets 8, 10, is of relatively solid form, specifically in the form of the solid cylinder and not merely a hollow cylinder. Multiple channels 50, 52, 54 for guiding the various media are then formed into the innermost bracket 6—irrespective of the rotational symmetry. The channels 50, 52, 54 each ultimately lead to one of the nozzles 12, 14, 16 in the various brackets 6, 8, 10. The channel 50 leads firstly to the innermost nozzle 12 and extends along the central axis Z. The other channels 52, 54 extend with a spacing to the central axis Z in the radial direction R and, in the present case, are inclined with respect to the central axis in order to realize the smallest possible dimensions toward the front and to ensure sufficient structural space for corresponding media connections 56 at the rear B. In this case, the media connections 56 are, for example, HPLC fittings.

In the embodiment shown here having three brackets 6, 8, 10, the channel 52 leads through the innermost bracket 6 and opens at the front F thereof, into a head space 58 which is formed in the axial direction A between the innermost bracket 6 and the middle bracket 8. In combination with the innermost nozzle 12, the head space 58 is annular. The second medium, which emerges from the front F of the innermost bracket 6 via said channel 52, enters the head space 58 and is guided there from the outside along the innermost nozzle 12 in order to ultimately emerge in annular fashion through the corresponding middle nozzle 14. The same applies analogously to the outermost nozzle 16, with the difference that the associated third medium does not emerge from the front F of the innermost bracket 6 but emerges from the side, that is to say in the radial direction R, and is firstly guided into an annular channel 60 in the middle bracket 8, from where the third medium is guided for example through one or more channels 62 in the axial direction A into a head space 64 between the middle bracket 8 and the outermost bracket 10. From there, the third medium is then, analogously to the second medium, guided in annular fashion between the middle nozzle 14 and outermost nozzle 16, and ultimately discharged at the front F.

The extrusion device 2 shown here has four operating modes. A first operating mode is extrusion operation, in which the hollow structure 4 is manufactured. A second operating mode is assembly operation, in which the individual brackets 6, 8, 10 of the extrusion assembly 38 together with shrink-fitted nozzles 12, 14, 16, that is to say nozzles fitted thermally by means of a transverse press fit, are inserted one inside the other in order to subsequently be able to be connected to the media lines and clamped in a tool magazine or directly in the tool change assembly 40. A third operating mode is then tool change operation, that is to say the fixing in the tool change assembly 40, during which operation the tool change assembly 40 is unlocked in order for the extrusion assembly 38 to be removed from a holder or magazine, fixed in the tool change assembly and connected to the drive unit 42. A fourth operating mode is fine adjustment operation, that is to say the fine adjustment of the nozzles 12, 14, 16 in the axial direction A in order to compensate for tolerances in the manufacturing process and in the fitting of the nozzles 12, 14, 16 into the brackets 6, 8, 10. For this purpose, the brackets 6, 8, 10 are axially moved or positioned such that all of the mouths 26 lie in one plane. In the fourth operating mode, the monitoring of the position may be performed for example by optical means. From there, the brackets can be moved according to the requirements for the extrusion operation.

As already indicated, at least two and in the present case even all of the brackets 6, 8, 10 are mounted on one another by means of in each case one bearing, which is formed from a precisely machined bearing bore, the internal wall 30 thus formed in the interior of the outer brackets 8, 10, and in each case two narrow, encircling bearing regions 18, 20, 22, 24 (bearing inner surface) on the external wall 31 of the inner brackets 6, 8. Bearing regions 18, 20, 22, 24 allow statically determinate and sufficiently rigid guidance in the bearing bore of the outer bracket 8, 10 in each case, with friction being minimized owing to the small areas of contact. The bearing regions 18, 20, 22, 24 are arranged one behind the other in the axial direction A; more specifically, each of the two bearings has a front bearing region 18, 20 and a rear bearing region 22, 24, and in each case one front bearing region 18, 20 and one rear bearing region 22, 24 are arranged one behind the other. Here, the bearing regions 18, 20, 22, 24 are part of the inner brackets 6, 8 and function, together with the internal walls 30 of the outer brackets 8, 10, as plain bearings.

The bearings are configured such that damage to the generally very fragile nozzles 12, 14, 16 is prevented to the greatest possible extent during the placement one inside the other. For this purpose, each bearing is designed and arranged such that, as the two brackets 6, 8, 10 are placed one inside the other, at least the front bearing region 18, 20 thereof engages before the inner nozzle 12, 14 in each case enters the shank region of the outer nozzle 14, 16 in each case. At the latest when the inner nozzle 12, 14 enters the drawn conical region of the outer nozzle 14, 16, both the front bearing region 18, 20 and the rear bearing region 22, 24 engage in order to provide sufficient guidance and rigidity of the bearing arrangement and avoid contact of the nozzles 12, 14, 16.

In the case of particularly small gap dimensions between the nozzles 12, 14, 16, in particular also in the shank region thereof, it may be necessary for both bearing regions 18, 20, 22, 24 of each bearing to already engage before the mouth 26 of the inner nozzle 12, 14 in each case enters the shank region of the outer nozzle 14, 16. This configuration is illustrated in FIGS. 4A-4B, where the geometries of brackets 6, 8, 10 and nozzles 12, 14, 16 are depicted. In this case, the middle bracket 8 has, for assembly operation, a maximum movement travel 68 relative to the innermost bracket 6 which must correspond at least to a length 78 of the outer nozzle 14. The spacing of the two bearing regions 18, 22 to one another in the axial direction A is chosen to be as large as possible owing to the stability and rigidity of the bearing arrangement, and, in conjunction with a projecting length 72 of the middle bracket 8 beyond the rear bearing region 22, defines the maximum movement travel 68, during assembly operation, of the middle bracket 8 relative to the innermost bracket 6 in the axial direction A. The same applies analogously to the brackets 8, 10 and the nozzle 16 in conjunction with the bearing regions 20, 24.

The front bearing region 18, 20 is arranged as close as possible to the front F. For easier assembly and to avoid jamming, the front bearing region 18, 20 is in each case divided into two parts by a narrow encircling groove.

During extrusion, fine adjustment and tool change operation, the brackets 6, 8, 10 are moved relative to one another only to such an extent that, at all times, both the front and the rear bearing region 18, 20, 22, 24 are engaged, such that maximum stability is ensured. By contrast, during assembly operation, the front bearing region 18, 20 firstly stabilizes the brackets 6, 8, 10 as early as possible as they are placed one inside the other, with the rear bearing region 22, 24 then engaging only at a later point in time as said brackets are placed further one inside the other.

In the embodiment shown here, the front bearing region 18 also functions as a seal with respect to the head space 58 situated in front thereof. Aside from suitable material selection, close tolerances and high surface qualities in this region to ensure the sealing action, the guidance of fluid is also used to improve the sealing action. The front bearing region 18 of the innermost bracket 6 is divided into two parts by an encircling groove, which has the aforementioned advantages during the assembly operation. The groove is additionally used for conducting the third medium (in this case the curing agent). For this purpose, the third medium is conducted via a radial transverse bore from the channel 54 into the groove. The middle bracket 8 has, on the internal wall 30, that is to say on the inside of the bearing bore that forms the bearing arrangement with respect to the innermost bracket 6, and close to the base of the bearing bore, an encircling groove in the external wall 31. The groove is also referred to as annular channel 60. Via this, the third medium is conducted into two eccentric and axially parallel bores 62, and from these into the head space with respect to the bracket 10. There, the third medium ultimately enters the outermost nozzle 16. With this configuration of the guidance of fluid, it can be ensured that the second medium (in this case the wall material), if it enters the annular channel 60 through the sealing gap 74 of the bearing region 18, comes together with the third medium (curing agent) in the annular channel 60. This results in the sealing gap 74 being sealed as a result of crosslinking of the second medium (biomaterial) by the third material (curing agent), and thus realizes the function of a reactive seal. This is of importance in particular in the case of the second medium (in this case the biomaterial), owing to the relatively high viscosity thereof in relation to the support medium and curing agent and the resulting relatively high pressures in the head space 58.

This guidance of fluid however limits the maximum movement travel for the extrusion operation to a few hundred micrometers to a few millimeters. More specifically, the movement travel is limited to the range in which there is still a sufficient area of sealing contact of the front bearing region 18 (interrupted by the encircling groove in the axial direction in front of and behind the annular channel) in the middle bracket 8. This is dependent on the width of the front bearing region 18 as a whole in the axial direction A, the width of the encircling groove and of the annular channel 60, and the material properties and tolerances of the sealing regions of the inner brackets 6, 8.

In the exemplary embodiment shown, the channel 50 for the first medium has a constriction 76 having a diameter that corresponds to an internal diameter 78 of the innermost nozzle 12, which is connected to the channel 50. The constriction 76 is in this case simply a constant reduced internal diameter as viewed in cross section along the central axis Z, such that a simple step is formed in the channel 50 as a transition to the constriction 76. By contrast, in a variant that is not shown, a constriction 76 is formed by a forwardly continuously tapering internal diameter of the channel 50.

The innermost bracket 6 furthermore has a shape feature for an automated tool change, in this case for example an encircling groove 80, in order to allow form-fitting, easily releasable accommodation in a magazine.

The tool change assembly 40 serves as a holder device for the extrusion assembly 38 during operation, and clamps the brackets 6, 8, 10 such that these are movable relative to one another in the axial direction A as required and are otherwise optimally fixed and centered. For this purpose, the tool change assembly 40 has a clamping device 82, 84, 86 for each bracket 6, 8, 10. Each clamping device 82, 84, 86 has a tension arm 88 and a compression arm 90 which peripherally engage around an associated bracket 6, 8, 10 and which are each tubular. In the case of the outer brackets 8, 10, the compression arm 90 is inserted into the tension arm 88, whereas in the case of the innermost bracket 6, the tension arm 88 is conversely inserted into the compression arm 90. Each outer bracket 8, 10 then has a suitable peripheral contour that is clamped between the tension arm 88 and the compression arm 90. This is however reversed in the case of the innermost bracket 6, where the tension arm 88 pulls the bracket 6 into the compression arm 90. For this purpose, a groove 92 is also formed on the innermost bracket 6 behind the peripheral contour thereof in order to allow the tension arm 88 to engage therein. The tension arm 88 and the compression arm 90 of each clamping device 82, 84, 86 are mounted on one another by means of a bearing 94 and are movable relative to one another in the axial direction A for the purposes of clamping the associated bracket 6, 8, 10. A movement of the compression arm 90 relative to the tension arm 88 for the purposes of clamping or releasing the associated bracket 6, 8, 10 is realized for example by means of compressed air. The clamping is performed by means of the clamping device 82, 84, 86 at the front F in this case, whereas the bearing 94 thereof is arranged between the middle and the rear side B.

In the embodiment shown here, the peripheral contour for clamping is formed by a cone 96 of an associated bracket 6, 8, 10, and the clamping device 82, 84, 86 has a corresponding internal cone 98. Here, in the case of the outer brackets 8, 10, the internal cone 98 is formed on the associated tension arm 88, and in the case of the innermost bracket 6, the internal cone is formed on the compression arm 90. Furthermore, each cone 96 of the outer brackets 8, 10 has a planar surface 100 which extends in the radial direction R, which is annular, and which serves as a stop for the compression arm 90. An angle 102 of the cone 96 and of the internal cone 98 relative to the central axis Z is selected such that it firstly does not have a self-locking action, but on the other hand effective centering is made possible.

The cone 96 of the innermost bracket 6 is formed in the axial direction A at the rear B with respect to the aforementioned groove 80 for the tool change. The cones 96 of the outer brackets 8, 10 are inclined oppositely to the cone 96 of the innermost bracket 6, such that, owing to an internal pressure in each of the head spaces 58, 64, the innermost bracket 6 and the outer brackets 8, 10 are forced in opposite directions and are thus clamped in the tool change assembly 40 by way of the cones 96.

Furthermore, in the embodiment shown here, the tool change assembly 40 comprises three integrated double-acting pneumatic cylinders for preloading the brackets 6, 8, 10. The pneumatic cylinders each have a compression-side cylinder chamber 116 and a tension-side cylinder chamber 118. The interposed pneumatic piston 120 having a dynamic seal is connected, in the case of the innermost bracket 6, to the associated tension arm 88 and, in the case of the outer brackets 8, 10, to the associated compression arm 90. By pressurizing the two cylinder chambers 116, 118 with compressed air, in a manner controlled for example by means of a 5/2 directional pneumatic valve, an exactly axially acting force is generated, and the brackets 6, 8, 10 are thus preloaded.

During tool change operation, it is firstly the case, proceeding from the locked state as shown in FIG. 5A, that the outer clamping devices 82, 84 are unlocked by virtue of the associated compression arms 90 and tension arms 88 being moved apart, whereby the outer brackets 8, 10 are released, as shown in FIG. 5B. The extrusion assembly 38 is then fixed only by the innermost clamping device 82. The extrusion device 2 then moves, by means of the machine kinematics of the bioprinter, to a tool magazine and pushes the extrusion assembly 38, at the groove 80 described above, into the holding clamp of the tool magazine. The final, innermost clamping device 82 is thereupon unlocked, as shown in FIG. 5C, such that the extrusion assembly 38 is fully released and is then held only by the holding clamp of the tool magazine. The tool change assembly 40 then moves in the radial direction R away from the tool magazine and thus releases the extrusion assembly 38. A correspondingly reversed procedure is followed in order to lock an extrusion assembly 38 in the tool change assembly 40.

As can be seen in FIGS. 1, 2 and 3, the clamping devices 82, 84, 86 of the tool change assembly 40 each have, in the region of the extrusion assembly 38, a lateral cutout 104 to allow lateral entry of the extrusion assembly 38 and of the media connections 56 into the tool change assembly 40 and thus also allow a tool change.

Here, the tension arms 88 swing freely and are fixed and positioned in the axial direction A by the drive unit 42. The relative mobility of the brackets 6, 8, 10 is also implemented in this way. The drive unit 42 is connected to the tension arms 88 at least of the outer clamping devices 84, 86. In particular in the embodiment assumed here having three brackets 6, 8, 10 and three nozzles 12, 14, 16 for a support medium at the inside, a wall medium in the middle and a curing agent at the outside, a relative movement of the innermost bracket 6 with respect to the outer brackets 8, 10 is performed regularly during operation, whereas a relative movement of the outermost bracket 10 with respect to the middle bracket 8 is required relatively seldom by comparison. Therefore, in the exemplary embodiment shown here, the drive unit 42 is configured as a linear axis having a shallow-gradient ball screw drive having a threaded spindle 106 and having a fixed nut 108 and a driven nut 110. Rotational fixing and linear guidance of the nuts 108, 110 is performed for example by means of a profiled rail guide 112. Drive is imparted for example by means of stepper motors 114 and belt drives (with the associated belts not being illustrated here). The fixed nut 108 is connected by means of a suitable connection to the middle bracket 8, whereas the driven nut 110 is connected by means of a suitable connection to the outer bracket 10. To now move the innermost bracket 6 relative to the other brackets 8, 10, only the threaded spindle 106 is driven. The relative positions of the other brackets 8, 10 with respect to one another are maintained in the process. By contrast, to move only the outermost bracket 10 relative to the other brackets 6, 8, the driven nut 110 is moved, with the relative positions of the other brackets 6, 8 with respect to one another again being maintained.

The extrusion device 2 as a whole is configured such that, by adding further corresponding brackets to the extrusion assembly 38, further nozzles can be added, coaxially positioned, moved and operated.

Correspondingly, further clamping devices are also added to the tool change assembly 40, and the drive unit 42 is upgraded, for example with additional driven nuts, motors and linear carriages. By means of such an expansion, it is then for example made possible to manufacture multi-layer microstructures for replicating relatively complex blood vessels.

The following is a summary list of reference numerals, and the corresponding structure used in the above description of the invention:
2 Extrusion device
4 Hollow structure
6 First/innermost/inner bracket
8 Second/inner/outer/middle bracket
10 Third/outermost/outer bracket
12 First/innermost/inner nozzle
14 Second/inner/outer/middle nozzle
16 Third/outermost/outer nozzle
18 Front bearing region
20 Front bearing region
22 Rear bearing region
24 Rear bearing region
26 Mouth
28 Spacing (between two mouths)
30 Internal wall (of a bracket), bearing outer surface (of a bearing)
31 External wall (of a bracket), bearing inner surface (of a bearing)
32 Lumen
34 Wall
36 Cell
38 Extrusion assembly
40 Tool change assembly
42 Drive unit
44 Branch
48 Receptacle (of a bracket), fit
50 Channel (for a first medium)
52 Channel (for a second medium)
54 Channel (for a third medium)
56 Media connection
58 Head space (between innermost and middle bracket)
60 Annular channel
62 Channel (axially parallel bores from annular chamber into the head space)
64 Head space (between middle and outermost bracket)
68 Movement travel
72 Projecting length
74 Sealing gap
76 Constriction
78 Length (of a nozzle)
80 Groove (for tool change)
82 First/innermost/inner clamping device
84 Second/inner/outer/middle clamping device
86 Third/outermost/outer clamping device
88 Tension arm
90 Compression arm
92 Groove (for tension arm)
94 Bearing (tension arm against compression arm)
96 Cone
98 Internal cone
100 Planar surface
102 Angle
104 Cutout
106 Threaded spindle
108 Fixed nut
110 Driven nut
112 Profiled rail guide
114 Stepper motor
116 Compression-side cylinder chamber
118 Tension-side cylinder chamber
120 Pneumatic piston
A Axial direction
B Rear side
F Front side
R Radial direction
S Gap dimension
Z Central axis

1. An extrusion device for manufacturing a hollow structure by coaxial extrusion of a plurality of media, the extrusion device comprising:
nozzles;
a bearing; and
a plurality of brackets, each of said brackets provided for one of said nozzles for extruding one of the media, said brackets extending in an axial direction along a central axis and disposed coaxially, for coaxial arrangement of said nozzles one inside another, wherein at least two of said brackets follow one another in a radial direction and are mounted on one another in the radial direction by means of said bearing, such that said at least two brackets are movable relative to one another in the axial direction to adjust relative axial positions of respective ones of said nozzles.

2. The extrusion device according to claim 1, wherein:
said plurality of brackets are at least three said brackets; and
said brackets are movable relative to one another in the axial direction for adjusting the relative axial positions of said nozzles relative to one another.

3. The extrusion device according to claim 2, wherein:
a first of the media is a support medium for filling a lumen of the hollow structure;
a second of the media is a wall material for forming a wall of the hollow structure; and
a third of the media is a curing agent for curing a second medium when merged with the second medium.

4. The extrusion device according to claim 1, wherein:
one of said brackets is an inner bracket;
another of said brackets is an outer bracket; and
said inner bracket is movable in the axial direction relative to said outer bracket to such an extent that said nozzle of said inner bracket protrudes relative to said nozzle of said outer bracket in order to pierce an existing hollow structure for forming a branch by integral formation of the hollow structure on the existing hollow structure.

5. The extrusion device according to claim 1, wherein:
said bearing serves for stabilizing and guiding said brackets and for coaxially aligning said brackets as they are placed one inside another during an assembly process; and
said bearing has at least one bearing region which is configured such that, as said brackets are placed one inside the other, said bearing region always ensures guidance such that, in the radial direction, a clearance of an inner nozzle of said nozzles does not in any region exceed a specified gap dimension in relation to an outer nozzle of said nozzles, so as to prevent damage to said nozzles during a placement one inside the other.

6. The extrusion device according to claim 1, wherein:
said bearing has two bearing regions, including a front bearing region and a rear bearing region; and
said front bearing region and said rear bearing region are disposed one behind another, and spaced from one another, in the axial direction such that, at all times during an extrusion process, both of said bearing regions bear load, in order to at all times ensure sufficient guidance and rigidity of said bearing and thus a coaxial alignment of said nozzles.

7. The extrusion device according to claim 1, wherein said bearing has a sealing gap, an annular channel and at least one bearing region which is divided into two parts by said annular channel for one of the media, such that another of the media, which enters said annular channel through said sealing gap of said bearing, is merged with the medium in said annular channel in order to seal off said sealing gap.

8. The extrusion device according to claim 1, wherein said brackets are rotatable relative to one another about the central axis in order to compensate concentricity tolerances of said nozzles.

9. The extrusion device according to claim 1, wherein each of said brackets has a receptacle for one of said nozzles, and each said receptacle is configured as a transverse press fit.

10. The extrusion device according to claim 1, wherein said nozzles are configured such that the hollow structure is manufactured with an internal diameter of at most 1 mm.

11. The extrusion device according to claim 1, further comprising a drive unit by means of which said brackets are movable relative to one another automatedly.

12. The extrusion device according to claim 1, further comprising a tool change assembly which, for each of said brackets, has a clamping device for holding and fixing an associated one of said brackets, wherein each said clamping device has a tension arm and a compression arm which peripherally engage around said associated bracket.

13. The extrusion device according to claim 1, wherein said nozzles are configured such that the hollow structure is manufactured with an internal diameter in a range from 10 μm to 200 μm.

14. A method for manufacturing a hollow structure by means of the extrusion device according to claim 1, which comprises the step of:
moving at least two of the brackets relative to one another in the axial direction in order to adjust the relative axial positions of respective ones of the nozzles.

15. A method of using the extrusion device according to claim 1 for biofabrication, which comprises the step of:
manufacturing a hierarchical vascular system using the extrusion device.
